(19) United States
(12) Patent Application Publication
(10) Pub. No.: US 2013/0011831 A1
(43) Pub. Date: Jan. 10, 2013

(54) Compositions And Methods For The Rapid Detection Of Legionella pneumophila

(76) Inventors: Jing Luo, Shanghai (CN); Hong Cai, Shanghai (CN); Jing Chen, Shanghai (CN)

(21) Appl. No.: 12/935,131

(22) PCT Filed: Mar. 25, 2010
(86) PCT No.: PCT/CN10/00367

§ 371 Date: Sep. 28, 2010

(51) Int. Cl. C07H 21/04; G01N 21/64; C12Q 1/68

(52) U.S. Cl. 435/6.11; 536/24.32; 536/24.33; 435/6.12; 536/23.1

(57) ABSTRACT

The present application describes compositions and methods useful for the rapid detection of Legionella pneumophila. The compositions include capture probes, amplification primers, primer sets and detection probes that comprise nucleic acid molecules that hybridize to L. pneumophila 23S rRNA or DNA encoding 23S rRNA target sequences. Also described are methods for detecting and/or quantifying the amount of L. pneumophila in a sample using real time PCR (rPCR) or revere transcriptase real time PCR (RT-rPCR).

### FIELD

This application relates to compositions and methods for the detection of Legionella pneumophila and more specifically to compositions and methods for the detection of 23S rRNA L. pneumophila target sequences.

### BACKGROUND

Legionella pneumonia causes Legionnaires disease and Pontiac fever in humans. It can be community acquired or nosocomial, and sporadic or epidemic in nature. The fatality rate can approach 50% in immuno-compromised patients. Legionella bacteria are also known to persist in moist environments such as water reservoirs, which facilitates the spread of disease through infected sources such as cooling towers or drinking-water distribution systems.

A number of methods have been described to detect Legionella bacteria. The “gold standard” procedure to identify L. pneumophila in water samples is cultural isolation [10]. The culture of bacteria using a specialized buffered charcoal yeast extract (BCYE) medium is sensitive and accurate but requires about two weeks for maximum recovery followed by bacteria identification using combination of colony morphology, gram staining and serologic testing [1]-13]. To overcome these limits, immunofluorescent methods [14-17] and molecular approaches that utilize PCR have been developed [18-26]. PCR-based approaches primarily target the 5S, 16S and 23S rRNA genes and the macrophage infectivity potentiator (mip) gene of L. pneumophila [1-9, 25,26]. Real-time PCR methods have also been described for the rapid detection of Legionella [27-31]. While commercial real-time PCR kits are available that target the DNA of the L. pneumophila mip gene, assays that are based on Legionella DNA detection may result in false positives as DNA will not be degraded as quickly as RNA after the death of bacteria in certain conditions.

Accordingly, there is a need for novel compositions and methods for detecting L. pneumophila.

### SUMMARY OF THE DISCLOSURE

The present disclosure provides compositions and methods useful for the detection of L. pneumophila in a sample that target 23S rRNA or DNA encoding 23S rRNA. The disclosure provides capture probes useful for isolating L. pneumophila nucleic acids as well as PCR primers useful for amplifying target sequences and detection probes that selectively bind to and facilitate detection of the target sequences. Also provided are methods that use real time polymerase chain reaction (rPCR) or reverse transcriptase real time polymerase chain reaction (RT-rPCR) for the detection of L. pneumophila. Optionally, the amount of L. pneumophila in the sample is quantified using real time PCR.

The nucleic acid molecules and methods disclosed herein are selective for L. pneumophila 23S rRNA of or DNA encoding 23S rRNA. As shown in Example 2, the amplification primers and detection probes are selective against non-Legionella microorganisms as well as against non-pneumophila Legionella species. Furthermore, as shown in Example 3 RT-rPCR with primers and detection probes that target 23S rRNA readily amplifies all 15 L. pneumophila serogroups with similar sensitivity.

The detection methods described herein are significantly faster than the “gold standard” culture method, which often requires 2 weeks to incubate and identify Legionellae. In addition, the present methods are generally more accurate and sensitive than methods that rely on immunofluorescence or radioimmunoassays.

Accordingly, in one embodiment there is provided an isolated nucleic acid molecule comprising a sequence with at least 85% identity to any one of SEQ ID NOS: 1 to 15, or the complement thereof. In one embodiment, the nucleic acid molecule comprises a sequence with at least 90%, 95% or 99% identity to any one of SEQ ID NOS: 1 to 15, or the complement thereof. In one embodiment, the nucleic acid molecule hybridizes to Legionella pneumophila 23S rRNA or DNA encoding 23S rRNA.

In another embodiment, there is provided a capture probe comprising a nucleic acid molecule comprising a sequence with at least 85% identity to any one of SEQ ID NOS: 1 to 6, or the complement thereof, and an affinity label. Optionally, the nucleic acid molecule comprises a sequence with at least 90%, 95% or 99% identity to any one of SEQ ID NOS: 1 to 6. In one embodiment, the affinity label is biotin. Optionally, the affinity label is conjugated to a 5′ end of the nucleic acid molecule. In one embodiment, the affinity label is conjugated to the nucleic acid molecule through a spacer molecule such as a nucleic acid.

In one embodiment, there is provided a primer suitable for amplification of Legionella pneumophila 23S rRNA or DNA encoding 23S rRNA comprising a sequence selected from SEQ ID NOS: 1, 7, 12, and 13. Also provided are primer sets comprising a forward and reverse primer suitable for the amplification of 23S rRNA or DNA encoding 23S rRNA target sequences. In one embodiment, the primer set comprises a first primer comprising at least 85% identity to SEQ ID NO: 1 and a second primer comprising at least 85% identity to SEQ ID NO: 7. In another embodiment, the primer set comprises a first primer comprising at least 85% identity to SEQ ID NO: 12 and a second primer comprising at least 85% identity to SEQ ID NO: 13.

A further embodiment includes an isolated nucleic acid molecule produced by PCR amplification of Legionella pneumophila 23S rRNA or DNA encoding 23S rRNA using the primer sets described herein. In one embodiment, the first and second primers have the sequences of SEQ ID NO: 1 and 7 and the isolated nucleic acid molecule produced by PCR amplification comprises the sequence of SEQ ID NO: 17. In another embodiment, the first and second primers have the sequences of SEQ ID NOS: 12 and 13 and the isolated nucleic acid molecule produced by PCR amplification comprises the sequence of SEQ ID NO: 16.

In one embodiment, there is provided a detection probe comprising: a) a nucleic acid molecule comprising a sequence with at least 85% identity to any one of SEQ ID NOS: 8, 9, 10, 11, 14 and 15, or the complement thereof, and b) a detectable label. In some embodiments, the detection probe hybridizes to a target sequence comprising 23S rRNA or DNA encoding 23S rRNA. In one embodiment, the detection probe hybridizes to a nucleic acid molecule produced by PCR amplification with one of the primer sets described herein. The detectable label can be a fluorophore or other label that produces a detectable signal. In one embodiment, the detection probe comprises a quencher that interacts with a fluorophore through Fluorescence Resonance Energy Transfer (FRET) or contact quenching. Optionally, the fluorophore is attached to the 5′ end of the nucleic acid molecule and the quencher is attached to the 3′ end of the nucleic acid molecule and the detection probe is suitable for use in real time PCR.

The present disclosure also provides kits useful for the detection of Legionella pneumophila. In one embodiment, the kit comprises at least one of the primer sets as described herein. In one embodiment, the kit comprises instructions for the detection of Legionella pneumophila. Optionally, the kits include one or more detection probes or capture probes as described herein.

In another embodiment, the disclosure provides a method for detecting the presence of Legionella pneumophila in a sample comprising:
a) providing a sample suspected of containing a target nucleic acid comprising Legionella pneumophila 23S rRNA or DNA encoding 23S rRNA;
b) contacting the sample with a primer set comprising
   i) a first primer comprising at least 85% identity to SEQ ID NO: 1 and a second primer comprising at least 85% identity to SEQ ID NO: 7, or
   ii) a first primer comprising at least 85% identity to SEQ ID NO: 12 and a second primer comprising at least 85% identity to SEQ ID NO: 13;
c) amplifying the target nucleic acid using the first and second primers to produce a target nucleic acid amplification product; and
d) detecting the presence of the target nucleic acid amplification product, wherein the presence of the target nucleic acid amplification product indicates the presence of Legionella pneumophila in the sample.

In one embodiment, the target nucleic acid is amplified using polymerase chain reaction (PCR) In one embodiment, the method also comprises contacting the sample with a reverse transcriptase to produce DNA encoding 23S rRNA. In one embodiment, the DNA encoding 23S rRNA is cDNA. In one embodiment, a primer comprising a sequence selected from SEQ ID NOS: 1, 13, 7 and 12 is used to prime the reverse transcription of 23S rRNA.

In one embodiment, the methods described herein use real time PCR. For example, in one embodiment the presence of the target nucleic acid product is detected using real time PCR. Optionally, the target nucleic acid product is detected concurrently with the amplification of the target.

In some embodiments, the presence of the target nucleic acid amplification product is detected by contacting the target nucleic acid amplification product with one or more detection probes as described herein. In one embodiment, the detection probes are labeled with a fluorophore and used in a real time PCR reaction to detect the presence of the target nucleic acid amplification product in a sample.

In one embodiment, the methods described herein comprise steps directed to sample preparation. For example, in some embodiments the samples are treated to isolate or separate nucleic acids from other components in the sample. Accordingly, in one embodiment the method also comprises:
mixing the sample with one or more capture probes; and
isolating nucleic acid sequences that bind to the capture probes to provide a sample that contains Legionella pneumophila 23S rRNA or DNA encoding 23S rRNA target nucleic acid.

In one embodiment, the capture probes comprise a nucleic acid molecule comprising at least 85% identity to a sequence selected from SEQ ID NOS: 1 to 6, or the complement thereof.

### BRIEF DESCRIPTION OF THE DRAWINGS

The invention will now be described in relation to the drawings in which:

FIG. 1 provides a standard curve for the detection of L. pneumophila cells by RT-rPCR according to Example 1.

FIG. 2 is a bar graph showing the sensitivity of the detection method described in Example 3 for L. pneumophila serogroups 1 to 15.

FIG. 3 shows the sensitivity for Set 1 and Set 2 RT-rPCR primer/probe sets as described in Example 4.

### DETAILED DESCRIPTION

The present description provides compositions and methods for the detection of Legionella bacteria, specifically Legionella pneumophila. The description also provides isolated nucleic acid molecules that are useful for the detection of target sequences comprising L. pneumophila 23S rRNA or DNA encoding 23S rRNA. In one embodiment, the DNA encoding 23S rRNA is reverse transcribed 23S rRNA.

In one aspect, the methods described herein comprise detecting the presence of L. pneumophila in a sample by detecting 23S rRNA target nucleotide sequences. In some embodiments, the methods include isolating, amplifying and/or detecting target nucleotide sequences comprising 23S rRNA or DNA encoding 23S rRNA. In one embodiment, the target sequence is detected using real time PCR (rPCR) or reverse transcriptase real time PCR (RT-rPCR).

In one aspect, the disclosure provides isolated nucleic acid molecules that hybridize to target sequences comprising 23S rRNA or DNA encoding 23S rRNA. In one embodiment, the isolated nucleic acid molecule comprises a sequence with at least 85% identity to any one of SEQ ID NOS: 1 to 15, or the complement thereof. In one embodiment, the isolated nucleic acid molecule comprises a sequence with at least 90%, 95%, or 99% identity to any one of SEQ ID NOS: 1 to 15, or the complement thereof.

As used herein, the term “isolated nucleic acid molecule” refers to a nucleic acid substantially free of cellular material or culture medium when produced by recombinant DNA techniques, or chemical precursors, or other chemicals when chemically synthesized. The term “nucleic acid” is intended to include DNA and RNA and can be either double stranded or single stranded.

The term “identity” as used herein refers to the percentage of sequence identity between two nucleic acid sequences. To determine the percent identity of two nucleic acid sequences, the sequences are aligned for optimal comparison purposes (e.g., gaps can be introduced in the sequence of a first nucleic acid sequence for optimal alignment with a second nucleic acid sequence). The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences (i.e., % identity=number of identical overlapping positions/total number of positions times 100%). In one embodiment, the two sequences are the same length. The determination of percent identity between two sequences can also be accomplished using a mathematical algorithm. A preferred, non-limiting example of a mathematical algorithm utilized for the comparison of two sequences is the algorithm of Karlin and Altschul, 1990, Proc. Natl. Acad. Sci. U.S.A. 87:2264-2268, modified as in Karlin and Altschul, 1993, Proc. Natl. Acad. Sci. U.S.A. 90:5873-5877. Such an algorithm is incorporated into the NBLAST and XBLAST programs of Altschul et al., 1990, J. Mol. Biol. 215:403. BLAST nucleotide searches can be performed with the NBLAST nucleotide program parameters set, e.g., for score=100, wordlength=12 to obtain nucleotide sequences homologous to a nucleic acid molecules of the present application. To obtain gapped alignments for comparison purposes, Gapped BLAST can be utilized as described in Altschul et al., 1997, Nucleic Acids Res. 25:3389-3402. When these programs, the default parameters of the respective programs (e.g., of NBLAST) can be used (see, e.g., the NCBI website). The percent identity between two sequences can be determined using techniques similar to those described above, with or without allowing gaps. In calculating percent identity, typically only exact matches are counted.

As used herein “hybridize” refers to the formation of non-covalent bonds between base pairs in complementary nucleic acid molecules. Hybridization and the strength of hybridization (i.e., the strength of the association between the nucleic acids) is influenced by such factors as the degree of complementarity between the nucleic acid molecules, stringency of the conditions involved, and the melting temperature of the formed hybrid.

As used herein “stringency” refers to the conditions, for example temperature, ionic strength, pH and the presence of other compounds, under which nucleic acid hybridizations are conducted. Under conditions of high stringency, nucleic acid base pairing will occur only between nucleic acid fragments that have a high frequency of complementary base sequences. Under conditions of low stringency, nucleic acid base pairing will occur between nucleic acid fragments that have a lower frequency of complementary base sequences.

For example, the following conditions may be employed to achieve stringent hybridization: hybridization at 5× sodium chloride/sodium citrate (SSC)/5×Denhardt's solution/1.0% SDS at Tm −5° C. for 15 minutes, followed by a wash of 0.2×SSC/0.1% SDS at 60° C. It is understood, however, that equivalent stringencies may be achieved using alternative buffers, salts and temperatures. The present disclosure also contemplates varying the hybridization conditions depending on the desired stringency of hybridization between nucleic acid molecules. Additional guidance regarding hybridization conditions may be found in: Current Protocols in Molecular Biology, John Wiley & Sons, N.Y., 1989, 6.3.1-6.3.6 and in: Sambrook et al., Molecular Cloning, a Laboratory Manual, Cold Spring Harbor Laboratory Press, 1989, Vol. 3.

### Preparation of Samples

As used herein “sample” refers to any biological or environmental sample suspected of containing L. pneumophila or nucleic acids derived from L. pneumophila. Examples of environmental samples include, but are not limited to, water samples, soil, slurries, debris, biofilms, samples from containers of aqueous fluids, airborne particles or aerosols and the like. Examples of biological samples include, but are not limited to, any tissue or material derived from a living or dead organism, including humans, such as respiratory tissue, exudates, lung biopsy, bronchioalveolar lavage, nasal swabs, sputum, blood (e.g., whole blood, blood serum, blood plasma etc), urine, synovial fluid, cerebrospinal fluid etc. The term “sample” as used herein includes processed samples, such as those obtained from passing an sample over or through one or more filters, by centrifugation, or treating or washing the sample such as by selective adherence of sample components to a medium, matrix or support.

Samples may be processed according to any number of preparation, treatment or concentration steps known in the art to either remove contaminants or concentrate suspected microorganisms or bacteria, including L. pneumophila. For example, samples may be processed by concentrating components from a larger sample volume or from a substantially aqueous mixture by filtration or trapping of airborne particles from an air sample or microbes from a water sample.

In some embodiments, the samples suspected of containing Legionella cells are treated to expose nucleic acids contained therein. For example, a sample may be treated in order to lyse cells and release intracellular components, including nucleic acids, in a sample. In some embodiments the sample is a solution and may include other components, such as enzymes, buffers, salts, detergents, etc.

In some embodiments, cells in a sample may be lysed by cell rupture using either non-mechanical or mechanical methods. Non-mechanical methods include, but are not limited to, chemical methods, thermal methods and enzymatic methods. Mechanical methods include ultrasonication disruption using a homogenizer, or a French press, decompression, and pulverization. Other methods known to a person of skill in the art for lysing cells are also contemplated by the present disclosure.

### Isolating Nucleic Acids and Target Capture

In some embodiments, the methods described herein include isolating nucleic acids from other sample components prior to detecting target nucleic acids comprising L. pneumophila 23S rRNA or DNA encoding 23S rRNA. Optionally, the nucleic acids are isolated using specific and/or non-specific methods.

Non-specific target capture methods include methods for generally isolating or separating nucleic acids contained in a sample from other sample components, such as proteins, lipids and/or cellular debris. Examples of non-specific target capture include the selective precipitation of nucleic acids, adhering nucleic acids to a support and washing to remove other sample components, or the use of other means to physically separate nucleic acids, including Legionella nucleic acids, from a sample mixture that contains other components. Additional non-specific target capture methods may separate RNA, including Legionella 16S rRNA or 23S rRNA, from DNA in a sample by using a random oligonucleotide probes that capture single-stranded RNA sequences.

TABLE 1
Capture probes for L. pneumophila*

| SEQ ID NO. | Sequence |
|---|---|
| 1 | ttcccatcgactacgctctt |
| 2 | tcctgcacatggctagat |
| 3 | ttcacccgagttctctca |
| 4 | uucccaucgacuacgcucuu |
| 5 | uccugcacauggcuagau |
| 6 | uucacccgaguucucuca |

*All sequences are presented 5′ to 3′

### Capture Probes and Specific Target Capture Methods

In one embodiment, Legionella rRNA or DNA encoding rRNA is isolated from other sample components by specifically hybridizing the Legionella nucleic acids to a capture probe to form a target nucleic acid:capture probe complex.

The present disclosure provides nucleic acid molecules suitable for use in capture probes that hybridize to target L. pneumophila sequences comprising 23S rRNA or the DNA encoding 23S rRNA. As used herein, the term “capture probe” refers to a composition comprising at least one nucleic acid sequence that selectively hybridizes to a complementary target sequence. Capture probes can be used to isolate target sequences in a sample by hybridizing nucleic acids contained in a sample to a capture probe to form a target nucleic acid/capture probe complex that can then be separated or isolated from other sample components. Optionally, the capture probes can be used for the preparation of samples prior to the detection of L. pneumophila using the compositions and methods described herein.

In one embodiment, the capture probes comprise a nucleic acid molecule comprising at least 85% identity to any one of the nucleic acid sequences shown in Table 1, or the complement thereof. In another embodiment, the capture probes comprise a nucleic acid molecule with at least 90%, 95% or 99% identity to any one of the nucleic acid sequences shown in Table 1, or the complement thereof. In one embodiment, the capture probes selectively hybridize to Legionella 23S rRNA or DNA encoding 23S rRNA. In one embodiment, the capture probes comprise a capture label. As used herein “capture label” refers to a moiety that specifically binds to a corresponding binding partner and allows for separation or isolation of the target nucleic acid/capture probe complex. The capture label can be attached to the nucleic acid sequence of the capture probe through covalent bonds or through non-covalent interactions such as hydrogen bonds, hydrophobic or ionic interactions or the formation of chelates or coordination complexes. The capture label can also be directly or indirectly attached to the nucleic acid sequence of the capture probe. For example, in one embodiment a spacer molecule separates the capture label and the nucleic acid sequence. In a further embodiment the spacer molecule is a nucleic acid sequence that is not complementary to Legionella target sequence. In one embodiment, the nucleic acid spacer sequence is a substantially homopolymeric 10 to 40 nt sequence (e.g., A10 to A40).

In one embodiment, the capture label comprises biotin and the corresponding binding partner is avidin/streptavidin. For example, in one embodiment, the capture label of the capture probe is a 5′ biotin tail together with a nucleotide spacer sequence that is not complementary to Legionella nucleic acid.

In one embodiment, the binding partner is affixed to a support such as a matrix or particle, which facilitates the isolation of the target nucleic acid:capture probe complex. As used herein “isolating” refers to removing or separating components from a sample to obtain a sample comprising target nucleic acid molecules. For example, in one embodiment, target nucleic acids are isolated from other sample components such as proteins, cell debris, enzymes, buffers etc. In one embodiment, the target nucleic acid:capture probe complex is bound to a support and the support is washed to remove sample components with a wash buffer.

In one embodiment, the binding partner of the capture label is affixed or conjugated to a support matrix or to particles free in solution. The support matrix or particles can be made from materials known in the art, including, but not limited to, nitrocellulose, nylon, glass, polyacrylate, mixed polymers, polystyrene, silane, polypropylene and/or metal. In one embodiment, the binding partner is affixed or conjugated to magnetically attractable particles. For example, in one embodiment the capture probe is a biotinylated capture probe that hybridizes to target sequences and binds to avidin or streptavidin linkages found on uniform monodisperse magnetic spheres.

As shown in Example 1, the capture probes described herein are useful for isolating target nucleic acid molecules from a sample prior to detection of specific target sequences. In one embodiment, target capture occurs in a solution phase mixture that contains capture probes that hybridize to Legionella 23S rRNA target nucleic acid under hybridizing conditions. The target nucleic acid:capture probe complex is then brought into contact with a support comprising the binding partner of the capture label, thereby affixing the target nucleic acid:capture probe complex to the support. In one embodiment, the support is then washed to remove other components from the sample without dissociating the target nucleic acid from the capture probe or the capture probe from the specific binding partner that is conjugated or affixed to the support.

A person skilled in the art will appreciate that varying the hybridization conditions will alter the specificity of the binding between a capture probe and the target sequence. In one embodiment, the capture hybridization occurs at 65 degrees Celsius for 10 minutes in SCC buffer followed by washing with 0.1×SSC buffer. In one embodiment, the conditions for the hybridization between the capture probe and the target sequence comprise a temperature in a range between the Tm of the capture probe, and 15 degrees Celsius above the Tm of the capture probe. For example, in one embodiment, the Tm of the capture probe is around 60 degrees Celsius and the hybridization temperature is between 60 degrees Celsius and 75 degrees Celsius.

In one embodiment, target nucleic acid:capture probe complex is directly mixed with amplification reagents, primers and/or detection probes for amplification and/or detection of the target sequence.

In another embodiment, the target nucleic acid:capture probe complex is dissociated to release the target nucleic acid from the complex. For example in one embodiment the target nucleic acid is removed from the capture probe by denaturing the complex at a temperature higher than the Tm of the capture sequence in a buffer solution. In one embodiment, the buffer solution is a low ionic strength solution such as de-ionized water.

### Amplification Primers

In one aspect, the present disclosure provides primers that are useful for amplifying target sequence comprising L. pneumophila 23S rRNA or the DNA encoding 23S rRNA. In one embodiment, the primers comprise a nucleic acid sequence selected from SEQ ID NOS: 1, 7, 12 and 13 as shown in Table 2.

In one embodiment, there is provided a primer set suitable for the amplification of L. pneumophila 23S rRNA or DNA encoding 23S rRNA. As used herein the term “primer set” refers to two or more primers that co-operate to produce a nucleic acid product in an amplification reaction. In one embodiment, the primer set comprises a first primer comprising at least 85% identity to SEQ ID NO: 1 and a second primer comprising at least 85% identity to SEQ ID NO: 7. In another embodiment, the primer set comprises a first primer comprising at least 85% identity to SEQ ID NO: 12 and a second primer comprising at least 85% identity to SEQ ID NO: 13.

In one embodiment, reverse transcriptase PCR (RT-PCR) or polymerase chain reaction (PCR) amplification with a primer set comprising a first primer comprising the sequence of SEQ ID NO: 12 and a second primer comprising the sequence of SEQ ID NO: 13 produces an amplification product of 186 nucleotides comprising the following sequence:

(SEQ ID NO: 16)
GATAGGTGGGAGGCTGTGAAGTGAGGACGCTAGTTCTCATGGAG
CCGCCCTTGAAATACCACCCTGTTGTTATTGAGGTTCTAACTTGGT
CCAGTAATCCTGGATGAGGACAGTGTATGATGGGTAGTTTGACTG
GGGCGGTCTCCTCCCAAAGAGTAACGGAGGAGCACAAAGGTACC
CTCGGTA

In another embodiment, amplification with a primer set comprising a first primer comprising the sequence of SEQ ID NO: 7 and a second primer comprising the sequence of SEQ ID NO: 1 produces an amplification product of 167 nucleotides comprising the following sequence:

(SEQ ID NO: 17)
GCATTGAGAAGTGTGCTGGAGGTATCAGAAGTGCGAATGCTGACA
TGAGTAACGATAATGTGGGTGAAAAGCCCACACGCCGGAAGTCC
CAGGTTTCCTGCACGACGTTAATCGGAGCAGGGTGAGTCGGCCC
CTAAGGCGAGGCTGAAGAGCGTAGTCGATGGGAA

In one embodiment, the primers described herein are useful for the reverse transcription of 23S rRNA to generate a cDNA molecule encoding a target sequence of 23S rRNA. In a further embodiment, the primers are useful for the reverse-transcriptase PCR of 23S rRNA. In one embodiment, the primers are useful for amplifying 23S rRNA using RNA amplification methods (such as RT-PCR) or 23S ribosomal DNA (rDNA) using DNA amplification methods (such as PCR). Other methods known to a person skilled in the art to amplify nucleic acids using the primers described herein such as Transcription Mediated Amplification (TMA), Nucleic Acid Sequence-Based Amplification (NASBA), or Loop-mediated isothermal Amplification LAMP are also contemplated by the present disclosure.

TABLE 2
Amplification primers and detection
probes for 23S rRNA of L.pneumophila*

| SEQ ID NO. | Sequence |
|---|---|
| 1 | ttcccatcgactacgctctt |
| 7 | gcattgagaagtgtgctgga |
| 8 | tgagtaacgataatgtgggtgaaaag |
| 9 | gagtaacgataatgtgggtgaaaag |
| 10 | ugaguaacgauaaugugggugaaaag |
| 11 | gaguaacgauaaugugggugaaaag |
| 12 | gataggtgggaggctgtgaa |
| 13 | taccgagggtacctttgtgc |
| 14 | gaaataccaccctgttgttattgag |
| 15 | gaaauaccacccuguuguuauugag |

*All sequences are presented 5′ to 3′

As used herein, amplifying a target sequence in a PCR reaction refers to the process of repeatedly denaturing and annealing forward and reverse primers to a nucleic acid template in the presence of a polymerase enzyme in order to extend the primers with a sequence complementary to the template in a thermocycling reaction to produce additional copies of a nucleic acid sequence as is generally known in the art (See Dieffenbach C W and G S Dveksler (1995) PCR Primer, a Laboratory Manual, Cold Spring Harbor Press, Plainview N.Y.). As used herein, the term “polymerase chain reaction” (“PCR”) refers to the method of K. B. Mullis U.S. Pat. Nos. 4,683,195 and 4,683,202, hereby incorporated by reference, which describe a method for increasing the concentration of a segment of a target sequence in a mixture of genomic DNA without cloning or purification. The length of the amplified segment of the desired target sequence is determined by the relative positions of the forward and reverse primers with respect to each other.

Amplification using PCR typically requires reagents necessary to carry out amplification such as a nucleic acid precursors (dCTP, dTTP etc.) a polymerase, primers, template, and buffer. As used herein, the term “primer” refers to an oligonucleotide, that is capable of acting as a point of initiation of synthesis when placed under conditions in which synthesis of a primer extension product that is complementary to a nucleic acid strand is induced, (i.e., in the presence of nucleotides and an inducing agent such as DNA polymerase and at a suitable temperature and pH).

The primers described herein are also useful for the reverse transcriptase PCR of a target sequence of L. pneumophila 23S rRNA. As used herein, “reverse transcriptase PCR (RT-PCR)” refers to the process of first reverse transcribing at least a portion of a RNA molecule into its DNA complement (complementary DNA, or cDNA) using the enzyme reverse transcriptase, and then amplifying the resulting cDNA using PCR or real-time PCR.

### Detection Probes

In another aspect, the present disclosure provides detection probes useful for the detection of target sequences comprising L. pneumophila 23S rRNA or DNA encoding 23S rRNA. As used herein, “detection probe” refers to a composition that hybridizes to a target sequence and directly or indirectly provides a signal indicative of the presence of a target nucleic acid. In one embodiment, the detection probes comprise a nucleic acid comprising a sequence with at least 85% identity to any one of SEQ ID NOS: 8, 9, 10, 11, 14 and 15 as found in Table 2. In one embodiment, the detection probes comprise a nucleic acid comprising a sequence with at least 90%, 95% or 99% identity to any one of SEQ ID NOS: 8, 9, 10, 11, 14 and 15. In one embodiment, the detection probes hybridize to L. pneumophila 23S rRNA or DNA encoding 23S rRNA. In one embodiment, the detection probes hybridize to an isolated nucleic acid produced by the PCR amplification of L. pneumophila 23S rRNA or DNA encoding 23S rRNA with the primer sets described herein.

In one embodiment, the detection probe comprises SEQ ID NO: 8 or SEQ ID NO: 9 and hybridizes to a nucleic acid molecule produced by amplification with a first primer comprising the sequence of SEQ ID NO: 7 and a second primer comprising the sequence of SEQ ID NO: 1. In one embodiment, the detection probe comprises SEQ ID NO: 8 or SEQ ID NO: 9 and hybridizes to a nucleic acid molecule comprising SEQ ID NO: 17.

In one embodiment, the detection probe comprises SEQ ID NO: 14 and hybridizes to a nucleic acid molecule produced by amplification with a first primer comprising the sequence of SEQ ID NO: 12 and a second primer comprising the sequence of SEQ ID NO: 13. In one embodiment, the detection probe comprises SEQ ID NO: 14 and hybridizes to a nucleic acid molecule having SEQ ID NO: 16.

In one embodiment, the detection probe comprises one or more detectable labels. As used herein “detectable label” refers to any molecule attached to a probe which facilitates the detection of the probe or a nucleic acid molecule comprising the probe. In one embodiment, the detectable label is covalently or non-covalently attached to a nucleic acid molecule. In some embodiments the detectable label may include a further nucleic acid molecule, peptide, luminescent compound, fluorophore, radiomolecule, redox label or antibody. In some embodiments, the detectable label is bound to a secondary label which is itself detectable. In one embodiment, more than one detectable label is attached to a probe. In one embodiment, a plurality of different detectable labels are attached to a probe.

The detection probes described herein can be used to detect the presence of a target molecule comprising L. pneumophila 23S rRNA or DNA encoding 23S rRNA through hybridization of the detection probes to the target as generally known in the art. For example, in some embodiments the detection probes described herein are useful for methods that include Northern hybridization, Southern hybridization, In Situ Hybridization (such as Fluorescence In Situ Hybridization (FISH) or Tissue or Cell In Situ Hybridization), DNA or RNA sequencing, real time PCR, RT-rPCR, as well as other DNA/RNA amplification or detection methods known in the art.

In one embodiment, the detection probes described herein are useful for real time PCR (rPCR) or reverse transcriptase real time PCR (RT-rPCR) methods to detect L. pneumophila 23S rRNA amplification products. As used herein, “real time PCR” refers to the simultaneous amplification and detection of a PCR amplification product. A number of technologies are known in the art to perform real time PCR including, but not limited to, TaqMan™ (Applied Biosystems), LightCycler™ (Roche Applied Science), as well as the use of molecular beacons or double stranded DNA dyes such as SYBR® Green.

In some embodiments, the detection of amplification products using rPCR involves a detection probe that selectively hybridizes to the target amplification product and causes a change in a detectable signal. For example, in one embodiment the detection probe comprises a fluorophore and hybridization of the detection probe to a target amplification product produces a measurable change in fluorescence. In one embodiment, “quenching” techniques such as Fluorescent Resonance Energy Transfer (FRET) and contact quenching are used to indicate the presence of a target nucleic acid with a hybridization probe. Quenching techniques involve energy transfer from one fluorophore, commonly known as a “donor”, to another fluorophore or non-fluorescent molecule commonly known as a “quencher”. Generally, quenching is sensitive to the distance between the donor and quencher moieties. Examples of fluorescent hybridization probes suitable for real time PCR that use FRET or contact quenching include adjacent probes (e.g. LightCycler™ probes), 5′-nuclease probes (e.g. TaqMan™ probes), molecular beacons and strand-displacement probes. (See for example, Marras S A E. (2006) Selection of fluorophore and quencher pairs for fluorescent nucleic acid hybridization probes. In Didenko V V (ed.), Fluorescent Energy Transfer: Nucleic Acid Probes and Protocols. Humana Press, Totowa, N.J., Vol. 335, pp. 3-16, hereby incorporated by reference).

In one embodiment, the methods described herein use real time PCR with detection probes based on TaqMan™ technology (Applied Biosystems). TaqMan™ probes are designed such that they anneal within a DNA region amplified by a specific set of primers. As the Taq polymerase extends the primer and synthesizes the nascent strand, the 5′ to 3′ exonuclease activity of the polymerase degrades the probe that has annealed to the template. Degradation of the probe releases the fluorophore from the 5′ end of the probe thereby separating the fluorophore from the quencher and preventing FRET and allowing fluorescence of the fluorophore. In one embodiment, fluorescence detected in a real-time PCR thermal cycler is directly proportional to the amount of fluorophore released and the amount of DNA template present in the sample undergoing real-time PCR. Real time PCR can therefore also be used to quantify the amount of target DNA in a sample.

As shown in the Examples 1 to 4, the primers and detection probes described herein are able to detect L. pneumophila accurately and sensitively using RT-rPCR or rPCR. Accordingly, in one embodiment there is provided a detection probe comprising a nucleic acid molecule comprising at least 85% identity to any one of SEQ ID NOS: 8, 9, 10, 11, 14 and 15, that is labeled at the 5′ end with a fluorophore and at the 3′ end with a quencher. Examples of fluorophores include, but are not limited to, 6-carboxyfluorescein (FAM), tetrachlorofluorescin (TET), HEX, Cy3, Cy5, TMR, ROX and Texas red. Examples of quenchers include, but are not limited to, dimethylaminoazosulfonic acid (Dabcyl), tetramethylrhodamine (TAMRA), dihydrocyclopyrroloindole tripeptide minor groove binder (MGB) and non-fluorescent quenchers such as Eclipse™ (Epoch Biosciences). A person of skill in the art will appreciate the need to match the fluorescence emission spectrum of the donor fluorophore with the absorption spectrum of the quencher.

Kits for the Detection of L. pneumophila

In one embodiment there is provided a kit useful for the detection of L. pneumophila that includes a composition comprising one or more of the nucleic acid molecules described herein. In one embodiment, the kit comprises a primer set as described herein. In a further embodiment the kit comprises at least one capture probe, amplification primer and/or detection probe as described herein. The kits may also include nucleotides, enzymes and buffers useful for PCR as well as reagents useful for detecting the PCR products. In one embodiment, the kits include reagents suitable for carrying out reverse transcriptase PCR such as reverse transcriptase enzyme. In one embodiment, the kit includes reagents suitable for carrying out real time PCR or reverse transcriptase real time PCR. The kits may also include suitable packaging and/or containers such as plastic resealable tubes or the like for holding primers or other regents. In one embodiment, the kids include detailed instructions for carrying out the methods of the invention.

Methods for Detecting L. pneumophila

According to one aspect of the disclosure, methods are provided for detecting the presence of L. pneumophila or nucleic acids derived from L. pneumophila in a sample.

In one embodiment, the method comprises providing a sample suspected of containing a target nucleic acid comprising L. pneumophila 23S rRNA or DNA encoding 23S rRNA. In one embodiment, the sample contains 23S rRNA and is contacted with primers and reverse transcriptase enzyme to generate cDNA encoding 23S rRNA. In one embodiment, the primers comprise at least 85% identity to any one of SEQ ID NOS: 1, 7, 12 or 13. In one embodiment, the primers comprise at least 90%, 95% or 99% identity to any one of SEQ ID NOS: 1, 7, 12 or 13. In one embodiment, the primers comprise at least 85%, 90%, 95% or 99% identity to any one of SEQ ID NOS: 1, 7, 12 or 13 and are useful for amplifying 23S rRNA or DNA encoding 23S rRNA.

In one embodiment, the sample is processed in order to remove contaminants and to provide a sample comprising L. pneumophila 23S rRNA or DNA encoding 23S rRNA. For example, the sample may be processed to isolate or concentrate microorganisms or cells from the sample. In another embodiment, the sample may be treated to lyse cells and release any nucleic acids contained therein. The samples may also be processed with non-specific target capture methods or specific target capture methods as previously described. In one embodiment, the sample is mixed with one or more capture probes in order to isolate nucleic acids that contain L. pneumophila 23S rRNA or DNA encoding 23S rRNA. In one embodiment, the capture probes comprise a nucleic acid molecule with at least 85% identity to a sequence selected from SEQ ID NOS: 1 to 6. In another embodiment, the capture probes comprise a capture label such as biotin.

In some embodiments, the sample is provided in a tube, PCR plate, or other suitable vessel or container for performing the methods described herein. In one aspect, the methods comprise contacting a sample with a primer set suitable for amplifying target 23S rRNA or DNA encoding 23S rRNA. In one embodiment, the primer set comprises a first primer comprising at least 85% identity to SEQ ID NO: 1 and a second primer comprising at least 85% identity to SEQ ID NO: 7. In another embodiment, the primer set comprises a first primer comprising at least 85% identity to SEQ ID NO: 12 and a second primer comprising at least 85% identity to SEQ ID NO: 13.

In another aspect, the method comprises amplifying a target nucleic acid comprising 23S rRNA or DNA encoding 23S rRNA using the primers described herein to produce an amplification product. In one embodiment, the target nucleic acid is amplified using PCR. In one embodiment, the primers are annealed at a temperature between 58 degrees Celsius and 62 degrees Celsius, between 59 degrees Celsius and 61 degrees Celsius, or at about 60 degrees Celsius.

In one embodiment, the method comprises amplifying a target sequence comprising a portion of the 23S rRNA or DNA encoding the 23S rRNA of L. pneumophila and detecting the corresponding amplification product. The presence of the amplification product indicates the presence of L. pneumophila in the sample. The amplification product may be detected using any method known to one of skill in the art, including but not limited to methods that utilize the detection probes described herein. In one embodiment, the method comprises the use of real time PCR (rPCR) or reverse transcriptase real time PCR (RT-rPCR) detect the presence of the amplification product.

In some embodiments, the amplification products are detected by contacting the amplified product with at least one detection probe that hybridizes or is specific for the amplified sequence. In one embodiment, the detection probe comprises a nucleic acid sequence that comprises at least 85% identity to any one of SEQ ID NOS: 8, 9, 10, 11, 14 or 15. In one embodiment, the amplification products are detected using real time PCR and the detection probes are labeled with a fluorophore at the 5′end and a quencher at the 3′end. In one embodiment TaqMan™ technology is used to detect the amplification products in a thermo cycler as is commonly known in the art.

### Internal Controls

Optionally, an internal control (IC) can be used to assure that samples are successfully amplified and/or detected according to the methods described herein. Accordingly, one aspect of the present description includes IC primers, probes and templates suitable for use with the methods described herein for detection of L. pneumophila.

In some embodiments, the IC can target an artificial template sequence or a natural template sequence in the sample. In other embodiments, the IC can be competitive (i.e. uses the same primers as the target detection sequence) or uncompetitive (i.e. uses different primers than the target sequence but exhibits a similar amplification efficiency).

In one embodiment, the IC is an artificial uncompetitive IC with a template made by combining mip gene sequence with lambda DNA sequence and transcripted into RNA. In one embodiment, the IC forward primer comprises the sequence CGGATACAGCAGGAACTGAAG (SEQ ID NO: 18), the internal control reverse primer comprises the sequence GCTTACCAGTCCGTCGAGAA (SEQ ID NO: 19) and the internal control detection probe comprises the sequence TGTACTTTCGTGCTGTCGCGGATCG (SEQ ID NO: 20). In one embodiment, there is also provided an IC capture probe having the sequence GCTTACCAGTCCGTCGAGAA (SEQ ID NO:21)

The above disclosure generally describes the present application. A more complete understanding can be obtained by reference to the following specific examples. These examples are described solely for the purpose of illustration and are not intended to limit the scope of the disclosure. Changes in form and substitution of equivalents are contemplated as circumstances might suggest or render expedient. Although specific terms have been employed herein, such terms are intended in a descriptive sense and not for purposes of limitation.

The following non-limiting examples are illustrative of the present disclosure:

### EXAMPLES

### Example 1

### Detection of L. pneumophila 23S rRNA Target Sequence

Capture probes, amplification primers and detection probes described herein were tested for the amplification and detection of L. pneumophila target sequence as follows:
1) A series of test samples were prepared with a concentration of Legionella pneumophila cells from 10⁰ to 10⁻⁹ in Page's saline buffer; samples were plated in cell culture for counting.
2) 9 ml lysis buffer was then added to 1 ml of each test sample of Legionella pneumophila cells and incubated overnight.
3) Lysates for each test sample were then filtered through PES 0.22 um˜0.45 um membrane.
4) 500 ul of filtered cell lysate from step 3 was mixed in an annealing mixture with a L. pneumophila capture probe consisting of 5′-biotin-(A)₁₄-TTCCCATCGACTACGCTCTT (SEQ ID NO:1)-3′, as well as an IC template and IC capture probe consisting of 5′-biotin-(A)₁₄-GCTTACCAGTCCGTCGAGAA (SEQ ID NO: 21)-3′ in SSC buffer. The mixture was heated at 65° C. for 10 min to allow target nucleic acids to anneal to the capture probes.
5) Magnetic beads (Magnesphere Paramagnetic Particles, Promega) were added into the annealing mixture for binding of target L. pneumophila onto the bead surface. The beads were then washed twice with wash buffer (0.1*SSC).
6) RNA was extracted by heating the beads at 65° C. for 10 min and captured nucleic acids were released into RNase free H2O.
7) Extracted RNA was then transferred into a PCR plate with a pre-aliquoted RT-rPCR mixture that included target amplification primers consisting of SEQ ID NOs. 1 and 7 and a detection probe comprising either SEQ ID NO: 8 or 9, as well as IC amplification primers consisting of SEQ ID NOs. 18 and 19 and a IC detection probe comprising SEQ ID NO: 20. RT-rPCR was then performed as follows on an ABI 7500 real time PCR system: (Step 1: Repeat1—42 C 5 min; Step 2: Repeat1—95 C 10 sec; Step 3: Repeat 40-95 C 5 sec, 60 C 30-34 sec.).

The internal control template was made by transcripting in RNA an artificial DNA sequence made by combining mip gene sequence with lambda DNA sequence. The resulting IC target sequence with primer and detection probe sequences underlined is as follows:

(SEQ ID NO: 22)
GTTAAACCTGGTAAATCGGATACAGCAGGAACTGAAGAATGCCAG
AGACTCCGCTGAAGTGGTGGAAACCGCATTCTGTACTTTCGTGCT
GTCGCGGATCGCAGGTGAAATTGCCAGTATTCTCGACGGACTGG
TAAGCCAGCTACTTTTCAGG

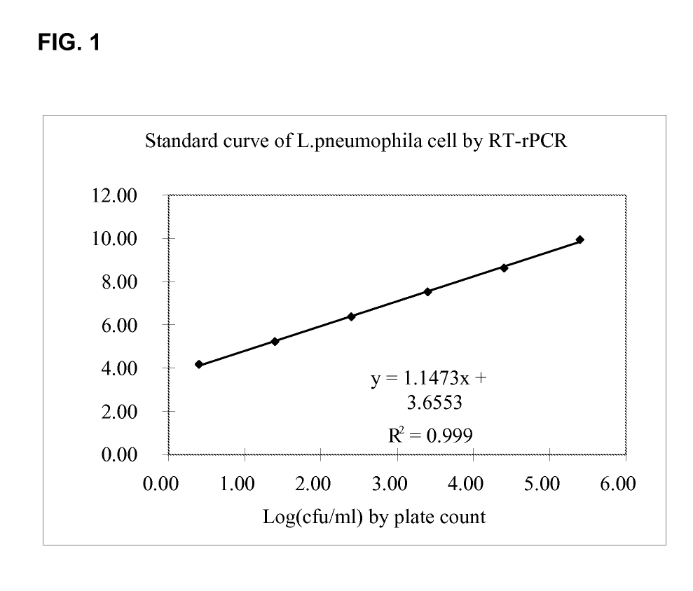

The RT-rPCR results using a detection probe comprising SEQ ID NO: 8 are presented in Table 3 and show a wide dynamic range from Log 10^{4.18} to Log 10^{9.94} LogCopy/assay. FIG. 1 shows a standard curve of the data presented in Table 3 for the detection of L. pneumophila by RT-rPCR with good linearity (R²=0.9984), indicating that the method is suitable for the quantitative detection of Legionella pneumophila.

TABLE 3
Quantification of L. pneumophila by RT-rPCR and Plate Count

| | RT-rPCR | Plate Count |
|---|---|---|
| Test sample | (LogCopy) | (cfu/ml) |
| Lysate-9 | # | 2.5\hat{ }10\hat{ }−1 |
| Lysate-8 | 4.18 | 2.5 * 10\hat{ }0 |
| Lysate-7 | 5.23 | 2.5 * 10\hat{ }1 |
| Lysate-6 | 6.37 | 2.5 * 10\hat{ }2 |
| Lysate-5 | 7.53 | 2.5 * 10\hat{ }3 |
| Lysate-4 | 8.62 | 2.5 * 10\hat{ }4 |
| Lysate-3 | 9.94 | 2.5 * 10\hat{ }5 |
| Lysate-2 | 11.40 | 2.5 * 10\hat{ }6 |

### Example 2

### Specificity Against Non-pneumophila Legionella species

The amplification primer and probe sets described herein were tested against 43 microorganism species that are either found in the same ecological region or phylogenetically close to Legionella for the detection of Legionella pneumophila. Set 1 consisted of forward and reverse amplification primers having SEQ ID NO: 12 or SEQ ID NO: 13 and a corresponding detection probe having SEQ ID NO: 14. Set 2 consisted of forward and reverse amplification primers having SEQ ID NO: 1 and SEQ ID NO: 7 and a corresponding detection probe having SEQ ID NO: 9 (Set 2).

RNA was extracted for each of the 43 non-pneumophila Legionella species as well as other non-legionella microorganisms listed in Table 4 using a Qiagen™ kit.

Each of the 43 species were then tested using RT-rPCR on RNA extracts at a concentration of 10̂7 copies/reaction with the same concentration of L. pneumophila serogroup 1 tested as a positive control. The detection probes were labeled at the 5′-end with a FAM fluorescent group and at the 3′-end with an Eclipse™ quencher. All primers & probes with fluorescent groups were synthesized by TAKARA Bio Inc.

RT-rPCR was performed in 50 μl reaction as set out in Table 4 using an ABI 7500 real time PCR system as follows: (Step 1: Repeat1—42 C 5 min; Step 2: Repeat1—95 C 10 sec; Step 3: Repeat 40-95 C 5 sec, 60 C 30-34 sec).

TABLE 4
Composition of RT-rPCR reaction mix.

| | Reagent | Volume (ul) |
|---|---|---|
| 1 | 2x One step RT-PCR buffer III | 25 |
| 2 | Takara Ex Taq HS (5 U/ul) | 1 |
| 3 | PrimeScript RT Enzyme MixII | 1 |
| 4 | Forward Primer (100 uM) | 0.1 |
| 5 | Reverse Primer (100 uM) | 0.1 |
| 6 | Detection Probe (100 uM) | 0.2 |
| 7 | ROX reference DyeII (50x) | 1 |
| 8 | Total RNA | 18 |
| 9 | Rnase free H2O | 3.6 |
| | Total | 50 |

As shown in Table 5, RT-rPCR using Set 2 did not detect any non-Legionella species or non-pneumophila species of Legionella and therefore exhibits excellent specificity to L. pneumophila. Set 1 did not detect any non-Legionella species and gave positive results for only a small number (11) of Legionella species. Both Set 1 and Set 2 positively detested samples of L. pneumophila.

TABLE 5
RT-rPCR testing of non-pneumophila Legionella species.

| | Non-pneumophila Legionella | | |
|---|---|---|---|
| No. | species microorganism | Set 1 | Set 2 |
| 1 | Legionella anisa | + | − |
| 2 | Legionella birminghamis | − | − |
| 3 | Legionella bozemanii 1 | − | − |
| 4 | Legionella bozemanii 2 | − | − |
| 5 | Legionella cherrii | + | − |
| 6 | Legionella cincinnatiensis | − | − |
| 7 | Legionella dumoffii | + | − |
| 8 | Legionella erythra 1 | − | − |
| 9 | Legionella feeleii 1 | − | − |
| 10 | Legionella feeleii 2 | − | − |
| 11 | Legionella gormanii | + | − |
| 12 | Legionella hackeliae 1 | − | − |
| 13 | Legionella hackeliae 2 | − | − |
| 14 | Legionella iordanis | − | − |
| 15 | Legionella lansingensis | − | − |
| 16 | Legionella longbeachae 1 | + | − |
| 17 | Legionella longbeachae 2 | + | − |
| 18 | Legionella maceachernii | − | − |
| 19 | Legionella micdadei | − | − |
| 20 | Legionella oakridgensis | − | − |
| 21 | Legionella parisiensis | + | − |
| 22 | Legionella sainthelensi 1 | + | − |
| 23 | Legionella sainthelensi 2 | + | − |
| 24 | Legionella tucsonensis | + | − |
| 25 | Legionella wadsworthii | + | − |
| 26 | Aeromonas hydrophila | − | − |
| 27 | Alcaligenes faecalis | − | − |
| 28 | Bacillus subtilis | − | − |
| 29 | Burkholderia cepacia | − | − |
| 30 | Clostridium d | − | − |
| 31 | Clostridium p | − | − |
| 32 | Enterobacter aerogenes | − | − |
| 33 | Escherichia coli | − | − |
| 34 | Flavobacterium | − | − |
| 35 | Klebsiella oxytoca | − | − |
| 36 | Listeria monocytogenes | − | − |
| 37 | Mucor racemosus | − | − |
| 38 | Proteus vulgaris | − | − |
| 39 | Pseudomonas aeruginosa | − | − |
| 40 | Pseudomonas fluorescens | − | − |
| 41 | Pseudomonas putida | − | − |
| 42 | Serratia marcescens | − | − |
| 43 | Stenotrophomonas maltophilia | − | − |

(“+” = positive, “−” = negative)

### Example 3

### Sensitivity Against Different L. Pneumophila Serogroups

Amplification primers having sequences SEQ ID NO:1 and SEQ ID NO:7 and a detection probe having sequence SEQ ID NO:8 were tested on L. pneumophila serogroups 1 to 15 using the RT-rPCR method described herein. The ATCC strains that correspond to each serogroup are listed in Table 6.

TABLE 6
ATCC strains corresponding to L. pneumophila serogroups 1 to 15
(See Brenner et al. [32] and Brenner et al. [33]).

| | | | ATCC |
|---|---|---|---|
| Serogroup | Organism | Designations | Number |
| 1 | Legionella pneumophila | Philadelphia-1 | 33152 |
| | subsp. pneumophila | | |
| | Brenner et al. | | |
| 2 | Legionella pneumophila | Togus-1 [NCTC 11230] | 33154 |
| | subsp. pneumophila | | |
| | Brenner et al. | | |
| 3 | Legionella pneumophila | Bloomington-2 | 33155 |
| | subsp. pneumophila | | |
| | Brenner et al. | | |
| 4 | Legionella pneumophila | Los Angeles-1 | 33156 |
| | subsp. fraseri Brenner et | | |
| | al. | | |
| 5 | Legionella pneumophila | Dallas 1E | 33216 |
| | subsp. fraseri Brenner et | | |
| | al. | | |
| 6 | Legionella pneumophila | Chicago 2 | 33215 |
| | subsp. pneumophila | | |
| | Brenner et al. | | |
| 7 | Legionella pneumophila | Chicago 8 | 33823 |
| | Brenner et al. | [NCTC 11984] | |
| 8 | Legionella pneumophila | Concord 3 | 35096 |
| | subsp. pneumophila | [NCTC 11985] | |
| | Brenner et al. | | |
| 9 | Legionella pneumophila | IN-23-G1-C2 | 35289 |
| | subsp. pneumophila | [NCTC 11986] | |
| | Brenner et al. | | |
| 10 | Legionella pneumophila | Leiden 1 | 43283 |
| | subsp. pneumophila | [Le 1, NCTC 12000] | |
| | Brenner et al. | | |
| 11 | Legionella pneumophila | 797-PA-H | 43130 |
| | subsp. pneumophila | | |
| | Brenner et al. | | |
| 12 | Legionella pneumophila | 570-CO-H | 43290 |
| | subsp. pneumophila | | |
| | Brenner et al. | | |
| 13 | Legionella pneumophila | 82A3105 | 43736 |
| | subsp. pneumophila | | |
| | Brenner et al. | | |
| 14 | Legionella pneumophila | 1169-MN-H | 43703 |
| | subsp. pneumophila | | |
| | Brenner et al. | | |
| 15 | Legionella pneumophila | Lansing 3 | 35251 |
| | subsp. fraseri Brenner et | | |
| | al. | | |

The primers and probe were tested as follows:
1) Total RNA was extracted from samples of L. pneumophila serogroup 1 to serogroup 15 lysates using a Qiagen RNeasy™ kit.
2) RNA concentrations were tested using SYBR Green I dye. RNA samples were then diluted to a concentration of about 5.0E+05 copies (about 100 CFU) per assay.
3) Purified RNA corresponding to each of serogroups 1 to 15 was transferred to a PCR plate. RT-rPCR was performed in a reaction volume of 50 μl as set out in Table 4 using an ABI 7500 real time PCR system according to the following protocol: (Step 1: Repeat1—42 C 5 min; Step 2: Repeat1—95 C 10 sec; Step 3: Repeat 40-95 C 5 sec, 60 C 30-34 sec).
4) The LogCopy number per assay for each serogroup based on fluorescence detection was calculated as set out in Table 7 as follows. The concentration of each serogroup was calculated in column 2 using a standard curve of Sybrgreen fluorescence at 520 nm. The LogCopy (LogCO) number for each serogroup in column 3 was experimentally determined using RT-rPCR. An adjusted LogCO number was then calculated based on the estimated Copy Number per μg in column 4 (determined from the results in columns 2 and columns 3) to account for variations in each serogroup input sample.

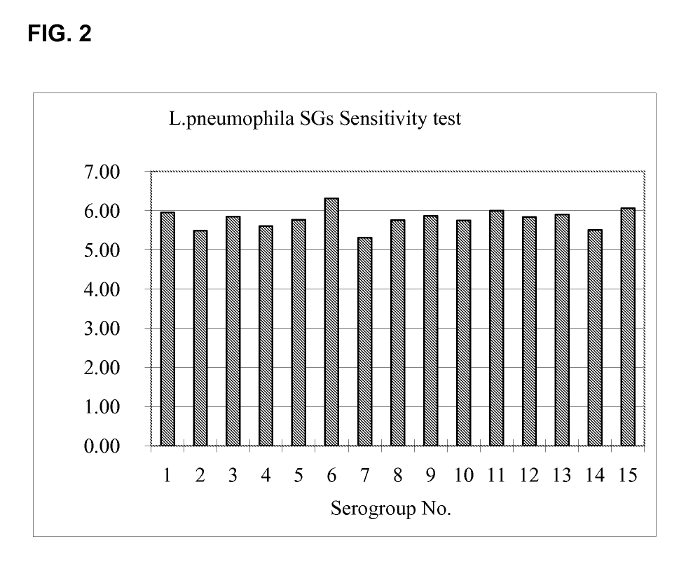

FIG. 2 shows the adjusted LogCopy number with respect to RT-rPCR for each of the 15 different serogroups. The results show that all L. pneumophila serogroups 1 to 15 were positively detected using the RT-rPCR method. Furthermore, similar sensitivity to each serogroup was observed and the variation of the LogCopy number for all serogroups was within 1 Log GU (genome unit).

TABLE 7
Calculation of adjusted LogCopy (LogCO) number for the
detection of L. pneumophila serogroups 1 to 15

| | 1 | 2 | 3 | | |
|---|---|---|---|---|---|
| | (y = 967.87x + 1390.1 | | LogCO | | 5 |
| | | Conc. | (RT- | 4 | Adjusted |
| Serogroup | Abs@520 nm | (ug/ml) | rPCR) | CopyNo./ug | LogCO |
| 1 | 6329 | 5.10 | 4.97 | 905467 | 5.96 |
| 2 | 8257 | 7.09 | 4.64 | 308489 | 5.49 |
| 3 | 11417 | 10.36 | 5.16 | 696026 | 5.84 |
| 4 | 8503 | 7.35 | 4.77 | 405016 | 5.61 |
| 5 | 7651 | 6.47 | 4.87 | 578215 | 5.76 |
| 6 | 12203 | 11.17 | 5.66 | 2056228 | 6.31 |
| 7 | 12570 | 11.55 | 4.67 | 202847 | 5.31 |
| 8 | 10545 | 9.46 | 5.03 | 570774 | 5.76 |
| 9 | 14062 | 13.09 | 5.28 | 730772 | 5.86 |
| 10 | 8245 | 7.08 | 4.90 | 560004 | 5.75 |
| 11 | 8858 | 7.72 | 5.19 | 993662 | 6.00 |
| 12 | 10016 | 8.91 | 5.09 | 694362 | 5.84 |
| 13 | 6839 | 5.63 | 4.95 | 790407 | 5.90 |
| 14 | 9406 | 8.28 | 4.72 | 319079 | 5.50 |
| 15 | 14526 | 13.57 | 5.50 | 1154907 | 6.06 |

### Example 4

### Use of Real Time PCR Reactions to Detect L. pneumophila

Probe and primer sets that included amplification primers SEQ ID NO:12 and SEQ ID NO:13 with probe SEQ ID NO:14 (Set 1) or amplification primers SEQ ID NO:1 and SEQ ID NO:7 with probe SEQ ID NO:9 (Set 2) were tested for the real time PCR detection of L. pneumophila as follows:
1) 6 samples of L. pneumophila Serogroup) DNA were prepared at concentrations of 10^{1.2}, 10^{2.2}, 10^{3.2}, 10^{4.2} and 10^{5.2} GU/assay.
2) Each primer and probe set was prepared and mixed with L. pneumophila DNA in a Taqman PCR master mix as set out in Table 8.
3) Real time PCR was performed separately using the Set 1 or Set 2 primers and probes with 3 trials for each set, at each level of DNA concentration (Taqman™ Univ. PCR Kit, Applied Biosystems). PCR conditions were as follows: (60 C for 2 min (1 cycle), 95 C for 10 min (1 cycle), 95 C for 15 seconds followed by 60 C for 1 min (50 cycles)).

TABLE 8
Composition of real time PCR (rPCR) reactions.
rPCR

| Reagent | Volume (ul) |
|---|---|
| Primer F (2 uM) | 2 |
| Primer R (2 uM) | 2 |
| Probe (2 uM) | 2 |
| DNA | 1 |
| Master Mix | 10 |
| DDW | 3 |
| Total | 20 |

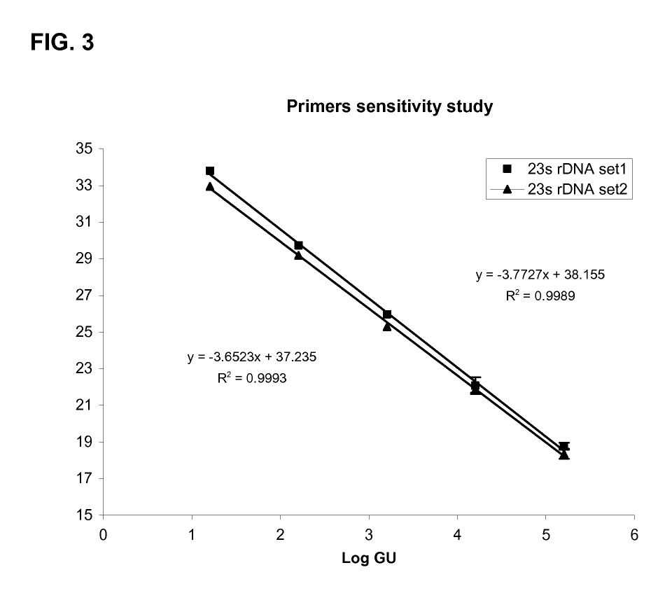

As shown in Table 9, real time PCR with Set 1 and Set 2 primers and detection probes showed comparable PCR efficiency (84.10%, 87.85%). FIG. 3 provides a graph of the real time PCR sensitivity (Ct (Cycle threshold) vs. Log GU) for each set for L. pneumophila DNA. The results in Table 9 and FIG. 3 show that the primers and probes described herein achieve a standard curve with good linearity and efficiency (within an acceptable range of 80%-120%), such as to be suitable for quantitative real time PCR.

TABLE 9
Real time PCR (rPCR) results for set 1 and set 2.

| | Set 1 | | Set 2 | |
|---|---|---|---|---|
| Log GU | Mean of Ct | STDV | Mean of Ct | STDV |
| 5.20 | 18.76 | 0.18 | 18.35 | 0.28 |
| 4.20 | 22.07 | 0.26 | 21.88 | 0.27 |
| 3.20 | 25.97 | 0.16 | 25.29 | 0.08 |
| 2.20 | 29.73 | 0.46 | 29.19 | 0.20 |
| 1.20 | 33.80 | 0.14 | 32.95 | 0.10 |

| | | |
|---|---|---|
| a | −3.7727 | −3.6523 |
| b (the lower the | 38.155 | 37.235 |
| better) | | |
| PCR efficiency = | 84.10% | 87.85% |
| 10^{(-1/a)-1} | | |

While the present disclosure has been described with reference to what are presently considered to be the preferred examples, it is to be understood that the application is not limited to the disclosed examples. To the contrary, the disclosure is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

1. An isolated nucleic acid molecule comprising a sequence with at least 85% identity to any one of SEQ ID NOS: 1 to 15, or the complement thereof.

2. The isolated nucleic acid molecule of claim 1 comprising a sequence with at least 90%, 95% or 99% identity to any one of SEQ ID NOS: 1 to 15, or the complement thereof.

3. The isolated nucleic acid molecule of claim 1, wherein the nucleic acid hybridizes to Legionella pneumophila 23S rRNA or DNA encoding 23S rRNA.

4. A capture probe comprising:
a) a nucleic acid molecule comprising a sequence with at least 85% identity to any one of SEQ ID NOS: 1 to 6, or the complement thereof, and
b) an affinity label.

5. The capture probe of claim 4, wherein the affinity label comprises biotin.

6. The capture probe of claim 4, wherein the affinity label is conjugated to a 5′ end of the nucleic acid molecule through a spacer nucleic acid.

7. A primer suitable for the polymerase chain reaction (PCR) amplification of Legionella pneumophila 23S rRNA or DNA encoding 23S rRNA comprising a sequence selected from any one of SEQ ID NOS: 1, 7, 12, and 13.

8. A primer set suitable for amplifying 23S rRNA or DNA encoding 23S rRNA comprising:
i) a forward primer comprising at least 85% identity to SEQ ID NO: 1 and a reverse primer comprising at least 85% identity to SEQ ID NO:7, or
ii) a forward primer comprising at least 85% identity to SEQ ID NO: 12 and a reverse primer comprising at least 85% identity to SEQ ID NO:13.

9. An isolated nucleic acid molecule produced by PCR amplification of Legionella pneumophila 23S rRNA or DNA encoding 23S rRNA with one of the primer sets of claim 7.

10. The isolated nucleic acid molecule of claim 9 comprising a sequence with at least 85% identity to the sequence of SEQ ID NO: 16 or SEQ ID NO: 17.

11. A detection probe comprising:
a) a nucleic acid molecule comprising a sequence with at least 85% identity to any one of SEQ ID NOS: 8, 9, 10, 11, 14 or 15, or the complement thereof, and
b) a detectable label.

12. The detection probe of claim 11, wherein the probe hybridizes to the isolated nucleic acid molecule of claim 9.

13. The detection probe of claim 11, wherein the detectable label comprises a fluorophore.

14. The detection probe of claim 13, further comprising a quencher that interacts with the fluorophore through Fluorescence Resonance Energy Transfer (FRET).

15. The detection probe of claim 14, wherein the fluorophore is attached to the 5′ end of the nucleic acid molecule and the quencher is attached to the 3′ end of the nucleic acid molecule for use in real time PCR.

16. A kit for the detection of Legionella pneumophila comprising one of the primer sets of claim 8 and instructions for use thereof.

17. The kit of claim 16, further comprising one or more detection probes of claim 11.

18. The kit of claim 16 further comprising one or more capture probes of claim 4.

19. A method for detecting the presence of Legionella pneumophila in a sample comprising:
a) providing a sample suspected of containing a target nucleic acid comprising Legionella pneumophila 23S rRNA or DNA encoding 23S rRNA;
b) contacting the sample with a primer set comprising:
i) a forward primer comprising at least 85% identity to SEQ ID NO: 1 and a reverse primer comprising at least 85% identity to SEQ ID NO:7, or
ii) a forward primer comprising at least 85% identity to SEQ ID NO: 12 and a reverse primer comprising at least 85% identity to SEQ ID NO:13;
c) amplifying the target nucleic acid using the forward and reverse primers to produce a target nucleic acid amplification product:
d) detecting the presence of the target nucleic acid amplification product, wherein the presence of the target nucleic acid amplification product indicates the presence of Legionella pneumophila in the sample.

20. The method of claim 19, where in step c) the target nucleic acid is amplified using polymerase chain reaction.

21. The method of claim 19, further comprising contacting the sample with a reverse transcriptase to produce cDNA encoding 23S rRNA prior to amplifying the target nucleic acid.

22. The method of claim 19, wherein the presence of the target nucleic acid product in step d) is detected using real time PCR.

23. The method of claim 19, wherein step d) further comprises contacting the target nucleic acid amplification product with a detection probe of claim 11.

24. The method of claim 19, wherein step a) further comprises:
i. mixing the sample with one or more capture probes of any one of claims 4 to 6, and;
ii. isolating nucleic acid sequences that bind to the capture probes to provide a sample that contains Legionella pneumophila 23S rRNA or DNA encoding 23S rRNA target nucleic acid.
